(19) United States
(12) Patent Application Publication
(10) Pub. No.: US 2016/0051601 A1
(43) Pub. Date: Feb. 25, 2016

(54) COSMETIC AND PHARAMCEUTICAL APPLICATIONS OF LACTOBACILLUS PENTOSUS

(71) Applicant: GREENTECH, St Beauzire (FR)

(72) Inventors: Laurent RIOS, Auzat La Combelle (FR); David TROPEL, Vic Le Comte (FR); Jean-Yves Antonin BERTHON, Romagnat (FR); Laurent CHAISEMARTIN, Vic Le Comte (FR)

(21) Appl. No.: 14/784,816

(22) PCT Filed: Apr. 15, 2014
(86) PCT No.: PCT/FR2014/050914

§ 371 Date: Oct. 15, 2015

(30) Foreign Application Priority Data
Apr. 15, 2013 (FR) 13/53395

(51) Int. Cl. A61K 35/747; C12R 1/225; A61Q 19/00; C12N 1/06; A61K 9/00; A61K 8/99

(52) U.S. Cl. CPC A61K 35/747; A61K 9/0014; A61K 8/99; A61Q 19/00; C12N 1/06; C12R 1/225; A61K 2800/805; A61K 2800/596; A61K 2800/85

(57) ABSTRACT

The invention concerns a cosmetic or dermatological composition for topical use comprising, as an active ingredient, the combination of a culture supernatant and of a cell lysate, from a Lactobacillus pentosus culture, as well as an obtaining method.

### TECHNICAL FIELD

The invention concerns cosmetic and pharmaceutical use of the Lactobacillus pentosus microorganism.

### BACKGROUND

The skin is an organ in constant renewal which covers the body surface and isolates it from the external environment. The skin forms a protection against external agents such as chemical and mechanical attacks, temperature, infections, humidity and radiations. The skin is structured into two main compartments: the epidermis covering the skin surface, and the deep dermis.

Though the entire structure of the skin actively participates in the body's defense, the role of the epidermis is essential for preventing the loss of water and other components of the body to its external environment and for protecting the body from a variety of environmental aggressions. Its main function consists therefore in protecting the body from external threats by establishing physical, chemical, biochemical and immunological barriers against them, while maintaining some exchange capacity between the outdoor and indoor environments.

The epidermis is an epithelium subdivided into many layers or strata, from the basal layer just above the dermis, by crossing the granular and spinal layers, up to the high layer, the stratum corneum. The Stratum corneum is composed of dead cells having neither a plasma membrane nor a core, and which are collected in a structure called cornified envelope (or cornea). The cornified envelope is highly resistant and consists of structural proteins and lipids. Thus, the stratum corneum is the layer of the epidermis playing the major role in the physical barrier.

Indeed, the stratum corneum constitutes a waterproof barrier which prevents desiccation. It has to be properly hydrated to ensure its protective function and a normal desquamation and to remain flexible. For this, epidermis produces its own Natural Moisturizing Factor or NMF.

The NMF consists of many small molecules such as urea, amino acids, lactic acids, sugars, mineral ions. Some have hygroscopic properties explaining the capacity to fix water. Others as cholesterol provide a degree of fluidity and flexibility to which would be otherwise a rigid and fragile membrane system.

The NMF represents up to 20-30% of dry matter of the stratum corneum and helps it to remain hydrated. With age, the skin dryness increases due to a decrease of NMF. Similarly, the level of NMF components is reduced after washing skin with soap. A reduction in NMF leads to a dry skin and to a disturbance of its barrier function. The skin, less protected, becomes then much more sensitive to damages caused by irritant agents. The NMF is thus considered as the essential component of the regulation of epidermal homeostasis and there is a need to reinforce it.

### BRIEF SUMMARY

Herein, a biological solution is provided for the protection of the skin's barrier function and its repair following an external aggression.

Thus, the invention concerns a cosmetic or dermatological composition for topical use, of which active ingredient may allow feeding and maintaining the NMF. This active ingredient comes from a Lactobacillus pentosus culture. In the present description, Lactobacillus pentosus will be abbreviated L. pentosus.

L. pentosus is a lactic bacterium which is prevalent in the fermentation medium of green olive called Spanish green olive. This probiotic species, also present in the digestive tract, is known for its capacity to stimulate some immune mechanisms. For example, it is reported as promoting the secretion of salivary immunoglobulin A in elderly patients (Y. Kotani et al. 2011), also as a stimulant of type 1 immunity, giving it a role in the fight against some infections and allergies (S H Koizumi et al. 2008).

The authors of the present invention found that an extract of L. pentosus culture, in cutaneous application, has beneficial properties on the skin, of which some are completely unexpected. These were first observed through the determination of a parameter, transpidermic water loss (TEWL), which is a good indicator of the skin's barrier function. It is commonly used to assess skin damages caused by some chemical agents, some mechanical aggressions or pathological conditions such as eczema. This parameter was measured in a test that will be illustrated in the examples, and which reveals a real enhancement function of the barrier function of a culture extract, on a skin presenting a barrier disruption caused mechanically, using an adhesive tape.

By continuing their investigations, the authors highlighted that, in order to have the expected properties, said culture extract has to assemble a supernatant and a cell lysate of the culture. The invention therefore provides a cosmetic or dermatological composition for topical use, comprising, as an active ingredient, the combination of a culture supernatant and of a cell lysate, said supernatant and lysate from L. pentosus culture.

The authors found that this combination allows gathering proteins, peptides, polysaccharides and short chain amino and organic acids and more generally all compounds forming the bacterial cell and metabolites produced by L. pentosus, which, in combination, enhance the barrier function of the stratum corneum or accelerate recovery when it is altered, by feeding NMF.

The active ingredient of the invention may be obtained through different ways.

The supernatant and the cell lysate may come from the same culture; according to this variant, they may come directly from a culture medium: therefore the active ingredient is obtained by mixing all or part of said supernatant and all or part of the cell lysate; thus, it can appear in the form of a total culture extract, after said medium or said extract has been subjected to a lysis step. But preferably, the weight ratio of supernatant to lysate is greater than 1. The lysis conditions have to lead to the inactivation and the release of cellular constituents of at least part of the cells of the medium. The lysis may therefore be only partial, or total. These conditions fall within the general knowledge of those skilled in the art, but advantageously, they cause lysis of all the medium's cells. According to another variant, the supernatant and the cell lysate may be separated from the culture medium, possibly treated then assembled to obtain the active ingredient of the invention. By treating either the supernatant and/or the cell lysate, any optimizing operation aiming to enhance their quality for the desired properties, is comprised.

Culture media adapted to the growth of L. pentosus comprise generally yeast extracts, peptones, salts, inorganic or organic sources of phosphate, nitrogen and potassium, etc. as well as sugars; these media, commercially available, as well as growth conditions of this bacterium (pH, temperature, aeration, agitation, redox potential, duration) are well-known concepts by those skilled in the art. According to the invention, such medium may be developed by those skilled in the art, it may be used as commercially available or may then be modified, most often by modifying the concentration and/or the nature of the aforementioned ingredients, in order to promote L. pentosus development.

Within the extension of their works, the authors noticed that the active ingredient of the invention has in addition beneficial properties on the radiance of the complexion.

The complexion radiance is of a multifactorial origin. It is a weighted mixture of characteristics of the texture of the skin surface (for example smooth or rough), of its brightness, of its microcirculation and of its color. The assessment of the complexion radiance is generally made by the observation carried out by panels of experts.

An active ingredient of the invention also has an anti-inflammatory activity which results from its capacity to inhibit lipooxygenases type enzymes. The lipooxygenases (LOXs) are a family of dioxygenases with non heminic iron, representing key enzymes in the biosynthesis of leukotrienes which are assumed to play an important role in the pathophysiology of many inflammatory and allergic diseases. The products coming from catalyzed oxygenation by LOXs (hydroperoxy/hydroxy eicosatetraenoic acids, leukotrienes and lipoxins) are apparently involved in the development of psoriasis and more generally in the skin irritation.

It is shown from these properties that the invention provides a combination of a culture supernatant and of a cell lysate, from L. pentosus culture, this combination being intended to be used in the treatment, by topical application, of skin allergies and skin inflammatory diseases, such as psoriasis.

The invention further provides the cosmetic use of a combination of a culture supernatant and a cell lysate, from L. pentosus culture of as previously defined. Thus, this combination may be a total extract of L. pentosus culture or is likely to be obtained by any one of the exposed methods and/or illustrated in the present description. In cosmetic, it is used to reinforce the barrier role of stratum corneum, reduce irritation, reduce inflammation and/or enhance complexion radiance. In dermatology with topical use, it is intended to be used in the treatment of the irritated or inflammatory state of the skin.

Whether for a cosmetic or a dermatological application, by dermal route, the concentration of the active ingredient varies advantageously from 0.1 to 10% by weight with respect to the total weight of the composition.

The production of the active principle comprises the following steps:

For optimal production of L. pentosus, the bacterium is advantageously cultured, until an advanced stage of the exponential phase of the microbial growth, preferably in the stationary phase. A medium particularly adapted to obtain an effective active ingredient is selected from M20 and MRS media, marketed as well as these same media of which the concentration and/or the nature of ingredients may be modified to promote L. pentosusgrowth. The pellet and the supernatant are then recovered. The recovery step is conventionally carried out and the routine techniques in microbiology are suitable. Thus, the supernatant may be separated from the culture medium by filtration or centrifugation. The resulting microbial biomass is treated in order to obtain a cell lysate.

A lysate commonly refers to a material obtained from the destruction or dissolution of biological cells by a phenomenon called cell lysis phenomenon causing thus the release of the intracellular biological constituents naturally contained in the cells of the considered microorganism. Within the meaning of the present invention, the term lysate is equally used to designate the totality of the lysate as defined above or a fraction thereof, this lysate being crude or having undergone one or more treatment(s), these should not substantially affect properties of the lysate in an active ingredient of the invention. The implemented lysate is therefore formed by all or part of the intracellular biological constituents and of the constituents of the cell membranes and walls. Advantageously, a lysate used for the invention is constituted by the totality of the obtained lysate. Cell lysis may be accomplished by different technologies, such as osmotic shock, heat shock, ultrasound or oven centrifugation type mechanical stress.

The lysate and the culture supernatant are then mixed. The weight ratio of the supernatant to the lysate varies preferably from 1 to 50, preferably from 5 to 15. The obtained active ingredient may be implemented under different forms such as solution, an optionally lyophilized atomized or concentrated powder.

The compositions according to the present invention may be formulated under any galenic form appropriate to their administration. The compositions according to the present invention may thus be formulated in the form of cream, gel, lotion, milk, water-in-oil or oil-in-water emulsion, solution, ointment, sprayer, body oil, after shave lotion, soap, protective stick for lips, stick and pencil for makeup, aerosol, roll-on, stick, ball-point, powder, wipe, incorporation into liposomes type vectors, glycospheres, cyclodextrins, into chylomicrons, macro-, micro-, nano-particles as well as macro-, micro- and nanocapsules and also adsorption on powdered organic polymers, talc, bentonites and other mineral supports.

The compositions according to the present invention may also contain common adjuvants or additives in cosmetics, as for example antimicrobial agents or perfumes as well as extracting or synthesis lipids, gelling and viscosifying polymers, surfactants and emulsifiers, hydro- or liposoluble active ingredients, plant extracts, tissue extracts, marine extracts, synthesis active agents.

The compositions according to the present invention may also comprise other complementary active ingredients selected for their action, for example for slimming effect, anti-cellulite effect, firming effect, moisturizing effect, anti-age effect, brightening effect, effect on skin color, antimicrobial activity, antioxidant activity, anti-radical activity, healing effect, tightening effect, anti-ride effect, chelating activity, complexing and sequestering activity, soothing effect, concealer effect, anti-redness effect, emollient activity, hair conditioner effect, anti-dandruff activity, stimulating effect of hair growth, inhibiting effect of hair fall, hair sheathing effect, depilatory activity, activity limiting hair growth, activity participating in cellular renewal, activity modulating the inflammatory response, activity participating in maintaining the oval of the face, but also sun protection, anti-irritant activity, cell nutrition, cellular respiration, anti-seborrheic treatments, skin tonicity, hair protection.

When the compositions according to the present invention contain complementary active ingredients, these are generally present in the composition at a sufficiently high concentration so that they may exert their activity.

The compositions according to the present invention are preferably daily used and applied once or several times per day.

The compositions according to the present invention are very well tolerated, they do not have any toxicity and their application on the skin, for extended periods, does not involve any systemic effect.

The invention provides also an advantageous method for preparing a cosmetic or dermatological active ingredient for topical use, based on a L. pentosus culture medium. This method comprises the following steps:

Obtaining an L. pentosusculture until stationary phase,

Centrifuging the culture medium in order to obtain a culture supernatant and a microbial biomass,

Separating the supernatant from the biomass,

Carrying out a cell lysis of the biomass to obtain a cell lysate, and

Mixing the supernatant and the lysate.

Preferably, the weight ratio of supernatant to lysate varies from 1 to 50.

Of course, any complementary step, for example, of treatment of the supernatant and/or the biomass, well known to those skilled in the art, may be performed, to obtain an active ingredient of the invention.

The invention also concerns L. pentosus CNCM 1-4730 strain as filed on Apr. 4, 2013 in accordance with Budapest Treaty with the National Collection of Microorganisms Culture (NCMC).

The present invention is now illustrated, without limitation, through the following examples and the attached figure.

### BRIEF DESCRIPTION OF THE DRAWINGS

The figure illustrates the anti-inflammatory activity of a culture extract of the present invention, by analysis of the release and/or synthesis of Interleukine 8 (pg/ml) by stimulated reconstructed epidermis.

### DETAILE DESCRIPTION

### Example 1

### Culture of L. pentosus CNCM 1-4730

L. pentosus CNCM 1-4730 strain is produced in a fermentor of 80 L in the culture medium presented in Table 1. The medium is inoculated with a 3% inoculum (volume/volume) from an L. pentosus CNCM 1-4730 culture aged 24 h. The growth of L. pentosus CNCM 1-4730 strain is carried out at 30 ° C. with a 50 revolutions/min stirring without air supply, nor pH regulation. The culture is led until the stationary phase is 20 hours after its inoculation.

TABLE 1
Culture medium of L. pentosus CNCM I-4730
Culture medium

| | Concentration in g/l of |
|---|---|
| Ingredient | culture medium |
| Yeast extract | 15.00 |
| Glucose | 20.00 |
| Tween ® 80 | 1.08 |
| K₂HPO₄ | 2.00 |
| Sodium acetate | 5.00 |
| Ammonium citrate | 2.00 |
| MgSO₄ | 0.20 |
| MnSO₄ | 0.05 |

### Example 2

### Preparation of the Active Ingredient According to the Invention

The culture obtained under the conditions described in Example 1 is completely centrifuged continuously on Sharples-type centrifuge at 15000 revolutions/min in order to separate the microbial biomass and the culture supernatant. Then the cellular biomass and the supernatant are treated as follows.

Preparation of the Cell Lysate of L. pentosus CNCM 1-4730

The microbial biomass is recovered (900 g of pellet at 21-25% dry matter) then resuspended in 5 volumes of water and centrifuged at 4000 revolutions/minute for 30 minutes. After removal of water, the biomass is recovered. The thus washed biomass is diluted in a 2M H₂SO₄ solution with a 50/50 mass ratio. The preparation is heated at 100° C. for 1h30 min in order to carry out the lysis of the bacterial cells.

Preparation of the Supernatant

60 L of supernatant from the Sharples centrifugation above are filtered at 0.2 μm on a filter plate. The filtrate is recovered.

Preparation of the L. pentosus CNCM 1-4730 Extract

In order to obtain the culture extract according to the present invention, a volume of cell lysate of L. pentosus CNCM 1-4730 is mixed with 9 volumes of supernatant, the pH is adjusted to 4 with NaOH.

### Example 3

### Possible Formulation of the Active Agent Based on the Lactobacillus Pentosus CNCM 1-4730 extract Obtained According to Example 2

The active ingredient obtained in example 2 is formulated at pH 5.5 according to the following composition, in mass percentage:

| | |
|---|---|
| Water | 89.39 |
| SEPIGEL ™ 305 | 2.00 |
| LABRAFAC ™ CC | 5.00 |
| MICROCARE ™ PM4 | 1.00 |
| SILK&CARE | 0.50 |
| L. pentosus extract | 2.00 |
| NaOH 10% | 0.09 |
| Citric acid 10% | 0.02 |
| Total | 100.00 |

### Example 4

### Anti-Inflammatory Activity

### Anti-Inflammatory Activity Assessed In Vitro by an Inhibition Test of the Activity of 5-Lipoxygenase (LOX-5)

LOX-5 is an enzyme involved in the pro-inflammatory process by allowing the formation of inflammatory leukotrienes from the arachidonic acid.

The anti-inflammatory activity of a culture extract of the invention, obtained in Example 2, is assessed, in vitro, by its capacity to inhibit LOX-5. It is evaluated by spectrophotometry (at 233 nm), by inhibiting the transformation of the linoleic acid into hydroxyperoxylinoleic acid. The monitoring of the inhibition of LOX-5 activity by different concentrations of culture extract according to the present invention allows determining IC₅₀ of this extract, that is to say, the concentration of said culture extract necessary to induce an inhibition of 50% of the 5-LOX activity.

For this, in a reaction medium (3 mL) containing 2950 μL at of phosphate buffer (0.1 M, pH=7.4), 1000 units of LOX-5 enzyme (10 !IL) and 50 μM of linoleic acid (10 μL), are added 30 μL of culture extract according to the present invention at various dilutions (0-16.8-56-112 mg dry matter/ml of extract).

The results of this study allowed highlighting an anti-inflammatory activity of the culture extract according to the present invention, by inhibiting the 5-lipoxygenase activity with IC₅₀ of 0.674 mg dry matter/mL of extract.

Anti-Inflammatory Activity Assessed In Vitro on the Synthesis and the Release of Interleukine 8 (IL-8) by Stimulated Reconstructed Epidermis

Reconstructed epidermis were stimulated by phorbol 12-myristate 13-acetate (PMA), a pro-inflammatory agent. The stimulation of the reconstructed epidermis by PMA at 0.3 μg/mL for 24 h causes an inflammatory state and generates an important release and/or synthesis of IL-8.

The release and/or synthesis of IL-8 were assessed by the immune-enzymatic method ELISA, from culture supernatants of the reconstructed epidermis as follows:

untreated epidermis,

epidermis treated with PMA to mime the inflammatory state,

epidermis pretreated with dexamethasone (10⁻⁷M), a synthesis glucocorticoid hormone serving as control), then treated with PMA,

epidermis pretreated with culture extract according to the present invention at different concentrations (0.112 and 0.280 mg dry matter/mL of extract), then treated with PMA.

The pre-incubation of reconstructed epidermis with dexamethasone inhibited the release and/or synthesis of IL-8 by 2.4 times.

The pre-incubation of reconstructed epidermis with culture extract obtained in Example 2, at 0.112 mg/mL and 0.280 mg/mL, significantly decreased by 1.8 times and 2.4 times the release and/or synthesis of IL-8, respectively.

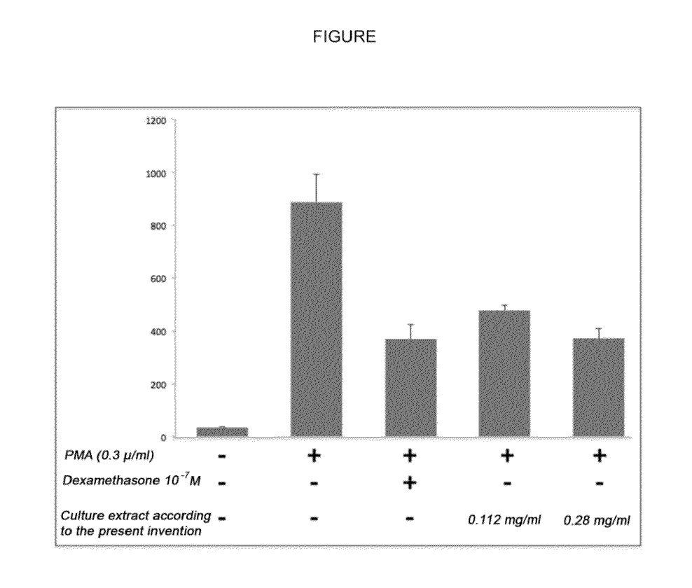

The results of this assay are presented in FIG. 1. They allowed highlighting a potent anti-inflammatory activity of culture extract according to the present invention. Indeed, culture extract according to the present invention at concentrations of 0.112 mg and 0.280 mg dry matter/mL of extract, inhibited the release and/or synthesis of IL-8 induced by the pro-inflammatory agent PMA. It is observed that this activity is near (for the concentration of 0.112 mg/mL) and is similar (for the concentration of 0.280 mg/mL) to that of dexamethasone.

### Example 5

### Protective Effect of the Skin Barrier Function

A clinical study was carried out in double blind on 8 human volunteers (8 Caucasian-type females aged 18 to 69) in order to evaluate, on the forearm, the protective effect of the skin barrier function of an active ingredient according to the present invention prepared according to Example 2.

Each volunteer applied, on one of its forearms, a cosmetic formulation containing 2% by weight of said active ingredient (namely 2 g of culture extract according to the present invention per 100 g of composition), and on its other forearm, a placebo cosmetic formulation (formulation with identical composition but which does not contain culture extract according to the present invention). The distribution of the forearms was carried out in a random way. Fifteen minutes after application of theses formulations, the skin barrier function of both forearms is altered by a method called <<tape-stripping>> method defined by 8 successive cycles, of 2 seconds each, of application/removal of an adhesive tape.

The assessment of skin barrier function is carried out before application of the formulations and 30 minutes after skin aggression through <<tape-stripping>>, by measuring the TransEpidermal Water Loss (TEWL) using a Tewameter™ TM 300 (marketed by Courage+Khazaka electronic). The measuring of TEWL allows evaluating the degree of water evaporation diffusing through stratum corneum (g.m⁻².h⁻¹). An alteration of skin barrier function induces a transepidermal water loss therefore an increase of TEWL. The lower TEWL is, the more the barrier function is preserved.

The results of this study are presented in table 2 below.

TABLE 2
Percentage of TEWL variation 30 minutes after skin aggression

| Placebo formulation | Formulation with culture extract at 2% |
|---|---|
| 13.8 ± 4 | 4 ± 0.7 |

The culture extract according to the present invention used at 2% in cosmetic formulation presents a protective effect of skin barrier function. Indeed, the transepidermal water loss (TEWL) measured 30 minutes after skin aggression is increased of only 4±0.7% in persons having received an application of the formulation containing culture extract according to the present invention, while this increase is of 13.8±4% in persons having received an application of the placebo formulation.

The culture extract according to the present invention presents a protective effect of skin barrier function.

### Example 6

### Repairing Effect of Skin Barrier Function

A clinical study was carried out in double blind on 14 human volunteers (14 Caucasian-type females aged 18 to 69) in order to evaluate, on the forearm, the repairing effect of skin barrier function (after skin aggression through <<tape-stripping>>) of an active ingredient according to the present invention. During 13 days (D−7 to D+6), twice a day, each volunteer applied, on one of its forearms, a cosmetic formulation containing 2% by weight of culture extract according to the present invention (namely 2 g of culture extract according to the present invention per 100 g of formulation), as prepared in Example 2; and on its other forearm a placebo cosmetic formulation (formulation with identical composition but which does not contain culture extract according to the present invention). The distribution of forearms was carried out in a random way. In D0, after 7 days of application of both types of formulations, the skin barrier function of both forearms is altered by <<tape-stripping>> (8 successive cycles, of 2 seconds each, of application/removal of an adhesive tape). The monitoring of skin barrier function is carried out on D0 after skin aggression, on D+1, on D+2 and on D+6, by measuring the TransEpidermal Water Loss (TEWL) using a Tewameter™ TM 300 (marketed by Courage+Khazaka electronic). The measuring of TEWL allows evaluating the degree of water evaporation diffusing through the stratum corneum (g.m⁻².h⁻¹). An alteration of the skin barrier function induces a transepidermal water loss thus an increase of TEWL. The lower TEWL is, the more the barrier function is preserved. The repairing effect of the skin barrier function is determined by monitoring the percentage of TEWL variation over time after skin aggression, taking as reference the measurement of TEWL carried out just after skin aggression. The repairing effect will correspond to a decrease over time of this percentage of TEWL variation.

The results of this study are presented in table 3 below.

TABLE 3
Percentage of TEWL variation over time after skin aggression

| | On D + 1 | On D + 2 | On D + 6 |
|---|---|---|---|
| Placebo formulation | 12.9 ± 3 | 8.5 ± 1.3 | −1.8 ± 0.3 |
| Formulation with 2% of culture | 5.9 ± 1.1 | −1.1 ± 0.3 | −5.4 ± 1.1 |
| extract according to the present | | | |
| invention | | | |

These results confirm that an active ingredient according to the present invention has a protective effect on skin barrier function because one day (D+1) after skin aggression, the percentage of TEWL variation increases only by 5.9±1.1% in persons having received the formulation containing culture extract according to the invention, compared to the increase of 12.9±3% in persons having received placebo formulation.

The culture extract according to the present invention accelerates and amplifies the recovery of skin barrier function. Indeed, from two days (D+2) after skin aggression, the percentage of TEWL variation is negative (−1.1±0.3%) meaning that the transepidermal water loss (TEWL) is less than that measured just after skin aggression (reference value). Six days (D+6) after skin aggression, the recovery of skin barrier function is 2.8 times more significant in persons having received the formulation containing the culture extract according to the present invention (percentage of TEWL variation of −5.4±1.1%)) with respect to persons having received placebo formulation (percentage of TEWL variation of −1.8±0.3%).

The culture extract according to the present invention accelerates and amplifies the recovery of the skin barrier function.

1. A cosmetic or dermatological composition for topical use, comprising, as an active ingredient, the combination of a culture supernatant and of a cell lysate, from a Lactobacillus pentosus culture.

2. The composition according to claim 1, wherein the weight ratio of supernatant to lysate in the combination varies from 1 to 50.

3. The composition according to claim 1, further comprising, as an active ingredient, a total extract of the culture, said extract having undergone a cell lysis.

4. The composition according to claim 1, wherein the concentration of the active ingredient varies from 0.1 to 10% by weight with respect to the total weight of the composition.

5. A cosmetic usemethod of using a combination of a culture supernatant and of a cell lysate, from a Lactobacillus pentosus culture, in order tecomprising reinforce-reinforcing the barrier function of stratum corneum.

6. The method according to claim 5, wherein the weight ratio of supernatant to lysate varies from 1 to 50.

7. The method according to claim 5, wherein of a total extract of Lactobacillus pentosus culture is applied.

8. A composition according to claim 1, for its useconfigured to reinforce barrier function of stratum corneum.

9. The use-method according to claim 5, further comprising reinforcing skin complexion radiance.

10. A dermatological composition for topical use according to claim 1, configured for treatment of irritated or inflammatory state of skin.

11. A combination of a culture supernatant and of a cell lysate, from a Lactobacillus pentosus culture configured for use in the treatment, through topical application, of skin allergies and inflammatory skin diseases.

12. A method of preparing a cosmetic or dermatological active ingredtientingredient for topical use, based on a Lactobacillus pentosus culture medium, comprising:
obtaining a Lactobacillus pentosus culture until stationary phase,
centrifuging the culture medium in order to obtain a culture supernatant and a microbial biomass,
separating the supernatant from the biomass,
carrying out a cell lysis of the biomass in order to obtain a cell lysate, and
mixing the supernatant and the lysate.

13. A Lactobacillus pentosus CNCM 1-4730 strain as filed on Apr. 4, 2013 in accordance with Budapest Treaty with the National Collection of Microorganisms Culture (NCMC).
